(19) United States
(12) Patent Application Publication
(10) Pub. No.: US 2023/0414690 A1
(43) Pub. Date: Dec. 28, 2023

(54) Full-Spectrum Plant-Derived Extracts Enriched In Total Phenols Including Monomers, Dimers, Trimers, Oligomers And Polymers, Method Of Manufacturing Same And Uses Thereof

(71) Applicant: Phenolics, LLC, Omaha, NE (US)

(72) Inventors: Michael S. Tempesta, El Granda, CA (US); F. Joseph Daugherty, Omaha, NE (US)

(73) Assignee: Phenolics, LLC, Omaha, NE (US)

(21) Appl. No.: 18/339,396

(22) Filed: Jun. 22, 2023

Related U.S. Application Data
(60) Provisional application No. 63/356,097, filed on Jun. 28, 2022.

(51) Int. Cl. A61K 36/45; A61K 36/185; A61K 36/736; A61K 36/87; A61K 36/73; A61K 36/31; A61K 36/63; A61K 36/752; A61K 36/48; A61K 36/899; A61K 36/39; A61K 36/38; A61K 36/81; A61K 47/40; A61K 47/12

(52) U.S. Cl. CPC A61K 36/45; A61K 36/185; A61K 36/736; A61K 36/87; A61K 36/73; A61K 36/31; A61K 36/63; A61K 36/752; A61K 36/48; A61K 36/899; A61K 36/39; A61K 36/38; A61K 36/81; A61K 47/40; A61K 47/12; A61K 2236/53; A61K 2236/51; A61K 2236/39; A61K 2236/55

(57) ABSTRACT

Methods for preparing compositions highly enriched in phenolic compounds from plant materials that naturally contain phenolic compounds such as anthocyanins and proanthocyanidins are enriched for a wide spectrum of phenolic compounds and consist of phenolic compounds including monomers, dimers, trimers, oligomers and polymers >3000 amu and over 35% concentrated total phenols. The compositions of the present disclosure which include phenolic compounds including monomers, dimers, trimers, oligomers and polymers >3000 amu and over 35% concentrated total phenols optionally include trisodium citrate and/or cyclodextrin.

### CROSS REFERENCE TO RELATED APPLICATION

This application claims priority to U.S. Provisional Patent Application 63/356,097.

### FIELD OF THE INVENTION

Plant-Derived Extracts Enriched In Total Phenols

### BACKGROUND OF THE INVENTION

Flavonoid compounds are present in all aerial parts of plants, with high concentrations found in the skin, bark, and seeds. Such compounds are also found in numerous beverages of botanical origin, such as tea, cocoa, and wine. The flavonoids are a member of a larger family of compounds called polyphenols. These compounds contain more than one hydroxyl group on one or more aromatic rings. The physical and chemical properties, analysis, and biological activities of polyphenols and particularly flavonoids have been studied for many years.

Anthocyanins are a particular class of naturally occurring flavonoid compounds that are responsible for the red, purple, and blue colors of many fruits, vegetables, cereal grains, and flowers. For example, the colors of fruits such as blueberries, bilberries, strawberries, raspberries, boysenberries, marionberries, cranberries, elderberries, etc. are due to many different anthocyanins. Over 700 structurally distinct anthocyanins have been identified in nature. Because anthocyanins are naturally occurring, they have attracted much interest for use as colorants for foods and beverages.

Recently, the interest in anthocyanin pigments has intensified because of their possible health benefits as dietary antioxidants. For example, anthocyanin pigments of bilberries (Vaccinium myrtillus) have long been used for improving visual acuity and treating circulatory disorders. There is experimental evidence that certain anthocyanins and other flavonoids have anti-inflammatory properties. In addition, there are reports that orally administered anthocyanins are beneficial for treating diabetes and ulcers and may have antiviral and antimicrobial activities. The chemical basis for these desirable properties of flavonoids is believed to be related to their antioxidant capacity. Thus, the antioxidant characteristics associated with berries and other fruits and vegetables have been attributed to their anthocyanin content.

Proanthocyanidins, also known as “procyanidins,” are another class of naturally occurring flavonoid compounds widely available in fruits, vegetables, nuts, seeds, flowers, and barks. Proanthocyanidins belong to the category known as condensed tannins. They are the most common type of tannins found in fruits and vegetables, and, are present in large quantities in the seeds and skins. In nature, mixtures of different proanthocyanidins are commonly found together, ranging from individual units to complex molecules (oligomers or polymers) of many linked units. The general chemical structure of a polymeric proanthocyanidin comprises linear chains of flavonoid 3-ol units linked together through common C(4)-C(6) and/or C(4)-C(8) bonds. Carbon-13 nuclear magnetic resonance (¹³C NMR) has been useful in identifying the structures of polymeric proanthocyanidins, and recent work has elucidated the chemistry of dimer, trimer and tetrameric proanthocyanidins. Oligomers and polymers of the flavonoid 3-ol units are predominant in most plants and are found with average molecular weights above 2,000 Daltons and containing 6 or more monomer units, Newman, et al., Mag. Res. Chem., 25:118 (1987). However, these larger oligomers and polymers can be entrapped and left behind in the various solvent extraction processes as well as subsequent chromatographic purification using a variety of solid supports.

Considerable recent research has explored the therapeutic applications of proanthocyanidins, which are primarily known for their antioxidant activity. However, these compounds have also been reported to demonstrate antibacterial, antiviral, anticarcinogenic, anti-inflammatory, anti-allergic, and vasodilatory actions. In addition, they have been found to inhibit lipid peroxidation, platelet aggregation, capillary permeability and fragility, and to affect enzyme systems including phospholipase A2, cyclooxygenase, and lipoxygenase. For example, proanthocyanidin monomers (i.e., anthocyanins) and dimers have been used in the treatment of diseases associated with increased capillary fragility and have also been shown to have anti-inflammatory effects in animals. See, e.g., Beladi, I. et al., Ann. N.Y. Acad. Sci. 284:358 (1977). Indeed, oligomeric proanthocyanidins have been described as useful components in the treatment of a number of conditions. Fine, A. M., Altern. Med. Rev. 5(2):144-51 (2000).

Proanthocyanidins may also protect against viruses. In in vitro studies, proanthocyanidins from witch hazel (Hamamelis virginiana) killed the herpes simplex 1 (HSV-1) virus. Erdelmeier, C. A., et al., Plant Med. June: 62(3):241-5 (1996); DeBruyne, T., et al., J. Nat. Prod. July: 62(7):954-58 (1999). Another study was carried out to determine the structure-activity relationships of the antiviral activity of various tannins. It was found that the more condensed the chemical structure, the greater the antiviral effect. Takechi, M., et al., Phytochemistry, 24:2245-50 (1985). In another study, proanthocyanidins were shown to have anti-herpes simplex activity in which the 50% effective doses needed to reduce herpes simplex plaque formation were two to three orders of magnitude less than the 50% cytotoxic doses. Fukuchi, K., et al., Antiviral Res., 11:285-298 (1989))

Recently, U.S. Pat. No. 10,772,901 to H. Shapland, et al., entitled “Medicinal composition for treating urinary tract infection (UTI)” has issued (the “'901 patent”). The '901 patent describes a pharmaceutical composition for use preventing or treating urinary tract infections (UTIs), chronic cystitis, overactive bladder, partial bladder obstruction or urethritis. Compositions of the '901 patent include one or more oligomeric tannins selected from proanthocyanidins and/or hydrolysable tannins, which are administered intravenously, intraurethrally, intravesically, and/or intrarenally. The '901 patent limits its claims to either oligomeric proanthocyanidins having a flavanol/flavan degree of polymerization of 4 to 25 or to oligomeric proanthocyanidins having an overall degree of polymerization of 4 to 20. This covers an approximate Molecular Weight range of 1200-7500 amu/1.2-7.5 kDa of those oligomeric proanthocyanidins. The '901 patent also describes other compositions in which hydrolysable tannins are bound to an anti-cancer agent and/or liposomes containing an anti-cancer agent.

The '901 patent notes at col. 3, lines 24-29 that “[i]n North America, cranberries, a source of tannins (e.g. proanthocyanidins), have long been considered to have medicinal properties, and cranberry juices has until fairly recently, been recommended for prevention and treatment of UTIs” but adds at col. 3, lines 33-40 that a recent Cochrane Database Systematic Review update “concluded that there is no evidence that oral consumption of cranberry juice decreases the number of symptomatic urinary tract infections in women”. This conclusion, as well as the absence of any teaching in the '901 patent to use its claimed compositions to treat UTIs or other malady via oral consumption, seems to teach away from the oral consumption of proanthocyanidins to treat or prevent UTIs or other maladies.

Other research, by A. Ledda, et al., “Highly standardized cranberry extract supplementation (Anthocran®) as prophylaxis in young healthy subjects with recurrent urinary tract infections,” Eur. Rev. for Med. and Pharmacol. Sciences, 2017; 21:389-93, reported on two groups of otherwise healthy patients between the ages of 12 and 18 who suffered from recurring UTIs. One group were given oral supplementation of one capsule of 120 mg of cranberry extract standardized to 36 mg proanthocyanidins for 60 days. The second group functioned as a control. It was reported that 63.1% of participants taking the oral supplement were asymptomatic with respect to UTIs, while 23.5% of the control subjects were asymptomatic. The report concluded that there was compelling evidence with respect to the efficacy of an oral supplementation based on the highly standardized Anthocran® cranberry extract as prophylaxis in young healthy subjects suffering from recurrent UTIs.

The degree of polymerization of phenolic compounds affects the ability of the molecules to permeate cell walls, an issue which has a direct effect on the efficacy of treatment via consumption of oligomeric phenolic compounds. There still is a need, therefore, for a method of producing an extract containing both low molecular weight phenolic compounds including monomers, dimers and trimers, as well as anthocyanins and proanthocyanidins having higher molecular weights without using very hot extraction temperatures which can cause degradation of the proanthocyanidins, and without using known ultrafiltration techniques which remove some of the low molecular weight polyphenolic material from the final product, while minimizing the known tendency of oligomeric phenolic compounds towards degradation.

### SUMMARY OF THE INVENTION

One aspect of the present disclosure comprises a highly enriched phenolic composition containing phenolic monomers, dimers, trimers, oligomers and a spectrum of phenolic compounds having degrees of polymerization ≥10, wherein the highly enriched phenolic composition contains at least 35% total phenols, preferably at least 40-50% total phenols and most preferably 50% or more total phenols including those phenolic compounds of greater than 2500 amu/2.5 kDa. Enriched, full-spectrum, polyphenolic extracts of the present disclosure derived from plant material may also be described as including phenolic monomers, dimers and trimers, together with phenolic oligomers selected from the group consisting of oligomers of from 1.2 kDalton through 2.4 kDalton and oligomers of from 2.4 kDalton through 3 kDalton, and polymers selected from the group consisting of polymers of from 3 kDalton through 6 kDalton, from 6 kDalton through 9 kDalton, and from 9 kDalton through 12 kDalton, wherein the polyphenolic extract contains >35% by weight total phenols, Alternatively described, the full-spectrum compositions of the present disclosure include plant-derived extracts enriched in total phenols comprising phenolic monomers, dimers, trimers, oligomers, and polymers. The compositions of the present disclosure may also be described as comprising phenolic monomers, dimers, trimers and a spectrum of polyphenolic oligomers and polymers >2500 amu or >2.5 kDa

These compositions of the present disclosure are useful as nutraceuticals and pharmaceuticals. For example, the compositions of this disclosure are useful as anti-infective (e.g., antiviral, anti-UTI and antimicrobial) agents and as anti-inflammatory agents.

When the highly enriched phenolic compositions of the present disclosure containing phenolic monomers, dimers, trimers and a spectrum of oligomers and polymers >2500 amu or >2.5 kDa are combined with trisodium citrate, the compositions are particularly useful as nutraceuticals and pharmaceuticals to be ingested orally.

When the phenolic compositions of the present disclosure containing phenolic monomers, dimers, trimers, and a spectrum of oligomers and polymers >2500 amu or >2.5 kDa are combined with cyclodextrin, the compositions are particularly useful as nutraceuticals and pharmaceuticals to be ingested orally.

Another aspect of the present disclosure provides for the extraction, isolation, and purification of compositions highly enriched in phenolic compounds and containing >35% weight by weight (“w/w”) total phenols which includes phenolic monomers, dimers, trimers, and oligomers, as well as polymers of ≥10 degrees of polymerization. The extraction, isolation, and purification methods of the present disclosure also may result in compositions highly enriched in phenolic compounds and containing >35% w/w total phenols which includes monomers, dimers, trimers and oligomers, together with polymers of ≥10 degrees of polymerization. The extraction, isolation, and purification methods of the present disclosure may also be described as resulting in a full-spectrum, polyphenolic extract containing >35% total phenols and at least 10% by weight of the polyphenolic extract are >2.5 kDalton.

More specifically, one aspect of this disclosure provides a method of preparing compositions highly enriched in phenolic compounds comprising: (a) providing a crude extract of one or more plant materials that contain phenolic compounds and polar non-phenolic compounds, (b) optionally filtering the crude extract, (c) contacting the crude extract with polymer resins selected from Mitsubishi SP-207 resin (referred to below as “SP-207”) or Lewatit OC 1064 resin (referred to below as “Lewatit 1064”), and the like, to individually, in combination or in series releasably adsorb phenolic compounds but not substantially retain polar non-phenolic compounds, (d) eluting polar non-phenolic compounds from the resins with an eluent, (e) eluting the resins with another eluent and collecting the resulting first fraction containing phenolic compounds; (f) eluting the resins with a second eluent and collecting a second fraction also containing phenolic compounds; (g) combining the fractions and then optionally repeating steps (c) through (g) to obtain a composition highly enriched in phenolic compounds containing least 35% w/w total phenols which includes monomers, dimers, trimers, oligomers, and polymers of ≥10 degrees of polymerization. The resulting compositions can also be described as being highly enriched in phenolic compounds and containing >35% w/w total phenols comprising monomers, dimers, trimers, oligomers, and polymers of ≥10 degrees of polymerization.

The foregoing and other features, utilities and advantages of the disclosure will be apparent from the following more particular descriptions of preferred embodiments of the disclosure and as illustrated in the accompanying drawings and as particularly pointed out in the appended claims.

### BRIEF DESCRIPTION OF THE DRAWINGS

The accompanying drawings, which are incorporated herein and form a part of the specification, illustrate non-limiting embodiments of the present disclosure, and together with the description, serve to explain the principles of the disclosure.

### In the Drawings

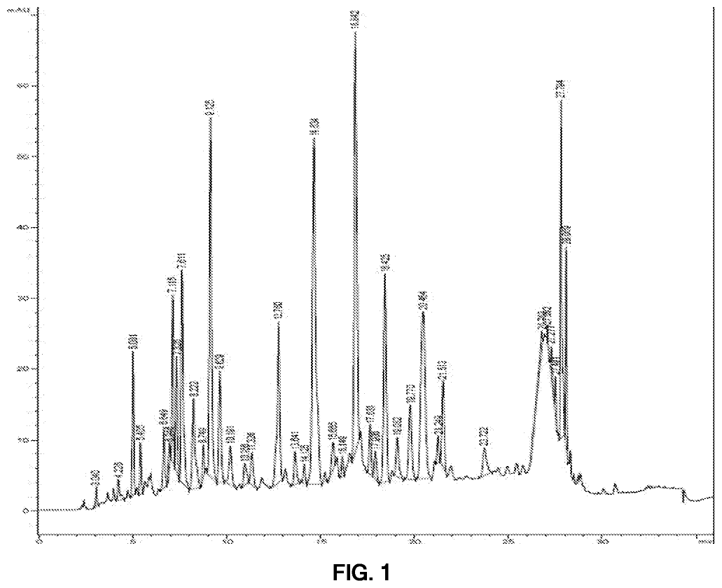

FIG. 1 is a High Pressure Liquid Chromatography (herein “HPLC”) chromatogram containing peaks representing distribution of different molecular weight polymeric procyanidins in a sample of cranberry extract;

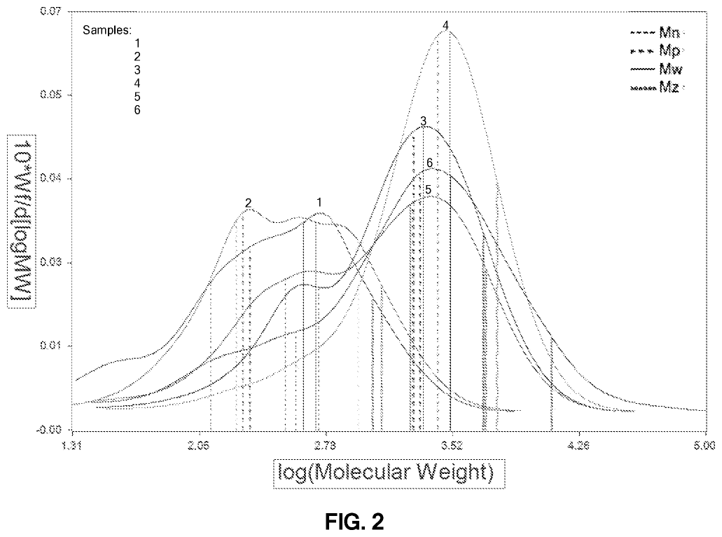

FIG. 2 is a graph containing six tracings labeled 1-6 illustrating the molecular weight distribution curves of the six samples described in Example 3;

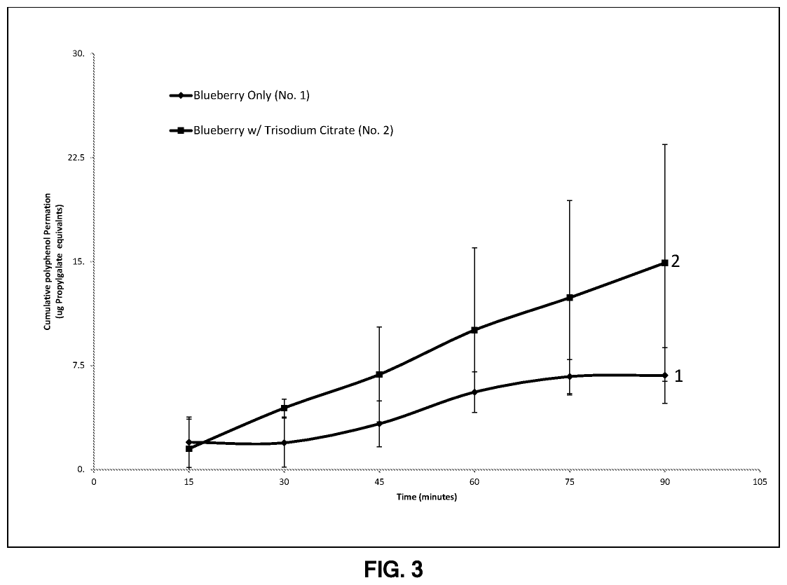

FIG. 3 is a graph containing two tracings labeled 1 and 2 illustrating the permeability of blueberry extracts though a MDCK monolayer in the presence and absence of 10 mM trisodium citrate as, described in Example 8;

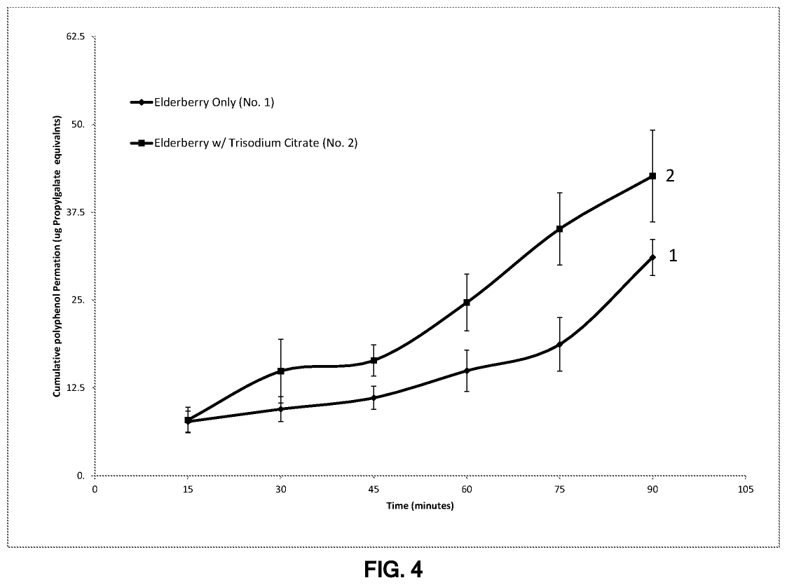

FIG. 4 is a graph containing two tracings labeled 1 and 2 illustrating the permeability of elderberry extracts though a MDCK monolayer in the presence and absence of 10 mM trisodium citrate, as described in Example 8;

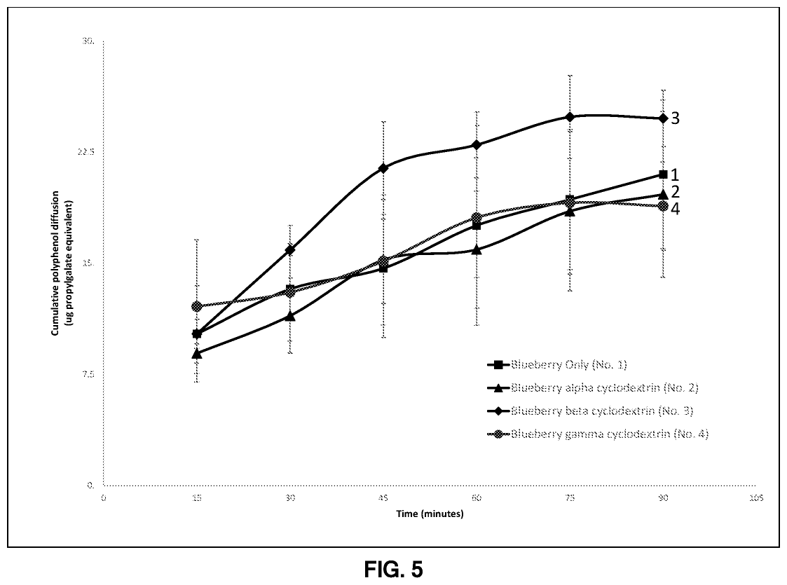

FIG. 5 is a graph containing four tracings labeled 1 through 4 illustrating the permeability of a blueberry extract through a CaCo2 membrane barrier alone and in combination with alpha, beta, or gamma cyclodextrin, as described in Example 9;

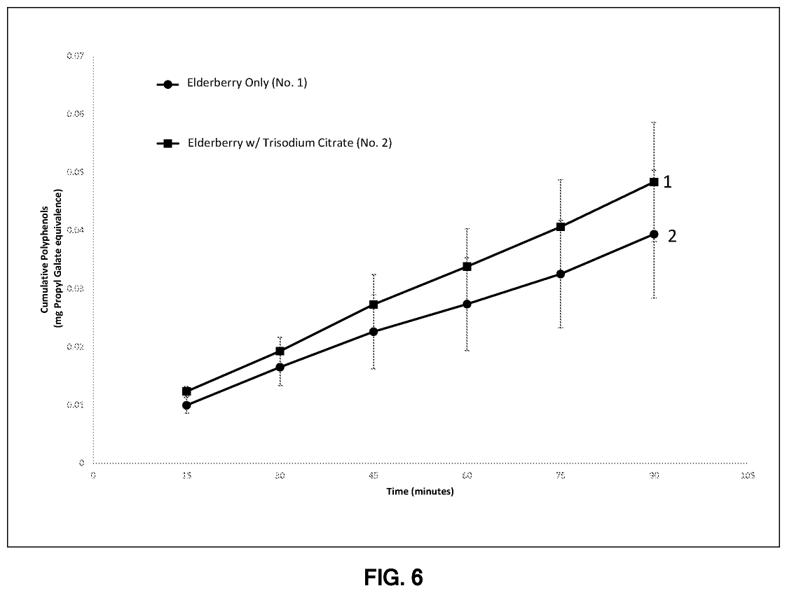

FIG. 6 is a graph containing two tracings labeled 1 and 2 illustrating the permeability of an elderberry extract through a CaCo2 membrane barrier alone and in combination with trisodium citrate, as described in Example 9; and

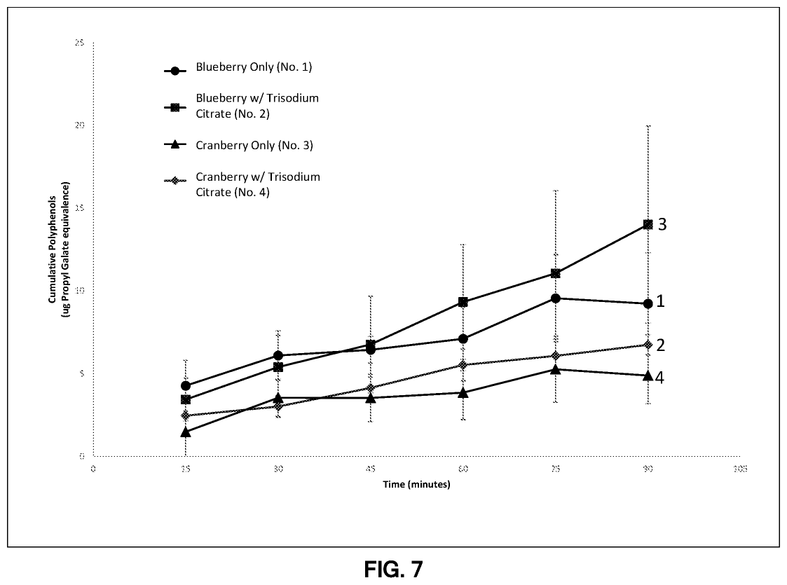

FIG. 7 is a graph containing four tracings labeled 1 through 4 illustrating and comparing the permeability of blueberry and cranberry extracts through a CaCo2 membrane barrier alone and in combination with trisodium citrate, as described in Example 9.

### DETAILED DESCRIPTION OF THE INVENTION:

This disclosure provides compositions highly enriched in phenolic compounds and methods for preparing compositions highly enriched in phenolic compounds from plant materials that naturally contain phenolic compounds such as anthocyanins and proanthocyanidins. The method of this disclosure further provides purified compositions enriched for a wide spectrum phenolic compounds. The term “phenol” and the term “phenolic compound” are used interchangeably herein and include monomers, dimers, trimers, and oligomers and polymers having one or more phenolic groups, and include, but are not limited to, anthocyanins, proanthocyanidins, and flavonoids, including such polymeric compounds having degrees of polymerization (“DP”) of ≥10 DP. As used herein, the term “highly enriched phenolic composition” refers to a composition enriched in phenolic compounds which includes monomers, dimers, trimers and oligomers having degrees of polymerization of ≥10 DP, which is equivalent to >3 kDa (kilodaltons) or >3000 amu (atomic mass unit) and comprising at over 35% w/w concentrated phenols and having substantially depleted levels of the major polar non-phenolic compounds present in crude extracts of plants, fruits, berries, and vegetables. Examples of such polar non-phenolic compounds include, but are not limited to, sugars, organic acids, cellulose, pectin, amino acids, proteins, nucleic acids, and water.

As used herein, dose means therapeutic dose or the amount of extract required when given orally to produce the desired outcome or benefit.

Highly enriched phenolic compositions are typically prepared from plant material extracts or concentrates. The term “extract” refers to a substance derived from a plant source that naturally contains phenolic compounds, including extracts prepared from the whole plant or from various parts of the plant, such as the fruits, leaves, stems, roots, bark, etc. The present method can use any source of phenolic compounds, most typically from botanically derived whole plant material or portions of the plant material such as the skins, peels, fruits, nuts, seeds, grain, foliage, stems, woody or fibrous material, and the like. Thus, the method of this disclosure is not limited to the particular part of the plant used to prepare the extract.

Most colored fruits, berries, and vegetables are known to contain phenolic compounds. Also, other suitable phenolic compound-containing plant materials that have a >0.5% total phenols based on dry weight that can be used in the methods of this disclosure include, but are not limited to, blueberries, bilberries, elderberries, plums, blackberries, strawberries, red currants, black currants, cranberries, cherries, red raspberries, black raspberries, grapes, hibiscus flowers, bell peppers, beans, peas, red cabbage, purple corn, violet sweet potatoes, olives, pomegranates, mangosteen, apples, hawthorn, gooseberries, acai, and oranges, including the whole plant material or the skins, peels, fruits, nuts, hulls, or seeds thereof. Plant sources containing <0.5% total phenols dry weight are excluded. The raw plant material may be used either as is (wet) or may be dried prior to extraction. Optionally, the raw plant material may be presorted by separating and removing the components low in anthocyanins and proanthocyanidins prior to extraction. Such a raw extract may then be initially processed as best described in U.S. Pat. No. 5,211,944. See also U.S. Pat. No. 7,306,815.

### EXAMPLE 1

In the development of the compositions of the present disclosure, research was conducted into determining the distribution of constituent phenolic compounds, from phenolic monomers, dimers and trimers, through oligomers of chain length 3-10 and polymers of chain length greater than 10 (herein “3mers”, “4mers”, “5mers”, “6mers”, “7mers”, “8mers”, “9mers”, “10mers” and “>10mers”). The method chosen for this determination of the weight % of the constituent phenolic compounds across the spectrum of the different degrees of polymerization of samples of super-enriched phenolic extracts of blueberries, cranberries, and elderberries obtained utilizing method steps 1-11 described herein (see Example 7), was the Folin-Ciocalteau Assay (herein “Folin-C”) detected all types of phenolic components, including both anthocyanins and procyanidins and all other related classes.

Currently, Folin-C is the simplest method available for the measurement of phenolic content in products. The Folin-C reagent can be prepared by dissolving 100 g sodium tungstate (VI) dihydrate and 25 g sodium molybdate (VI) dihydrate with 700 ml distilled water, 100 ml concentrated hydrochloric acid, and 50 ml of 85% phosphoric acid to which is added 150 g of lithium sulphate hydrate. For many years now, the Folin-C method of assay has been in use as a measure of polyphenols in natural products, with the basic mechanism being an oxidation/reduction reaction where the phenolic group is oxidized and a metal ion reduced.

In the present Example 1, to quantify by weight the constituent monomers, dimers, trimers, oligomers and polymers in an enriched phenolic composition containing 30% total phenols, dry powder samples of blueberry, cranberry and elderberry each standardized to contain 30% total phenols were reconstituted by dissolution in water. Then, utilizing the High Performance Liquid Chromatography (“HPLC”) methodology like that described in Robbins, J, et al., “Method performance and multi-laboratory assessment of a normal phase high pressure liquid chromatography-fluorescence detection method for the quantitation of flavonals and proanthocyanidins in cocoa and chocolate containing samples,” J. of Chromatography, A 1216.24 (2009) 4831-40, the samples were analyzed for constituent phenolic monomers, dimers, trimers, 4mers, 5mers, 6mers, 7mers, 8mers, 9mers, 10mers, and >10mers. These constituent phenolic compounds have molecular weight ranges of ±16 amu (approximately 10%) depending on the degree of oxidation of the “B” ring of the monomeric catechin or related unit. Although separately analyzed, the analytical results of the three tests are summarized below in Table 1:

TABLE 1

| | | micrograms/milliliter (μg/ml) | | |
|---|---|---|---|---|
| | | Blueberry | Cranberry | Elderberry |
| | amu/kDalton ± | 30% total | 30% total | 30% total |
| | 10% | phenols | phenols | phenols |
| Monomers | 300/0.3 | 3.11 | 5.11 | 3.66 |
| Dimers | 600/0.6 | 3.56 | 8.13 | 2.44 |
| Trimers | 900/0.9 | 3.13 | 6.18 | 3.09 |
| 4mers | 1200/1.2 | 2.77 | 4.70 | 2.93 |
| 5mers | 1500/1.5 | 3.51 | 4.80 | 2.39 |
| 6mers | 1800/1.8 | 5.23 | 5.97 | * |
| 7mers | 2100/2.1 | 3.42 | * | 4.49 |
| 8mers | 2400/2.4 | 3.76 | 8.33 | * |
| 9mers | 2700/2.7 | 6.29 | 9.91 | * |
| 10mers | 3000/3.0 | * | * | * |
| >10mers | >3000/>3.0 | 129.16 | 306.15 | 101.67 |

*below quantitation limit for the particular degree of polymerization.

It was the presence of such substantial portions of phenolic molecules having degrees of polymerization >10mers in extracts containing 30% total phenols (for blueberry 129.16 μg/ml, for cranberry 306.15 μg/ml, for elderberry 101.67 μg/ml) which was unexpected, the significance of which cannot be understated, because, it is these largest molecules which have some of the most significant biological activity, provided they can be extracted and neither overlooked nor intentionally left behind during extraction. These molecules are regularly left behind because in most separation techniques, the larger molecules of >10mers can be late eluting when using column or other separation techniques, and/or can bind/stick to resin or other surfaces. As a result, the >10mers fraction, which is approximately equivalent to >3 kDa or >3000 amu, especially the even higher molecular weight fractions, tend to be the least soluble and/or the least easily retrieved, and thus overlooked for treatment purposes.

It is believed by the inventors of the present disclosure that it in is the significant presence of the >3000 amu/>3 kDa polyphenols, when combined with a spectrum of monomeric and oligomeric polyphenols, that maximum efficacy of phenolic biological activity can occur. This is due to the outstandingly strong bioactivity of the polyphenols together with presence of phenolic monomers, dimers, and trimers, due to their ability to permeate cell walls.

### EXAMPLE 2

A highly-enriched dried cranberry extract containing approximately 36% total phenols was dissolved in water to a concentration of 5 mg/ml. HPLC analysis was then conducted under the conditions summarized below in Table 2, with the method's time gradient summarized below in Table 3:

TABLE 2

| | |
|---|---|
| Run time | 36 minutes |
| Flow Rate/Temperature | 1.00 mL/min/40° C. |
| Solvent A/B | Water + 5% CH₃CN + 0.1% TFA/CH₃CN |
| Injection vol. | 5 μL with needle wash |
| Λ | 210 nm, 275 nm, 310 nm, 550 nm, ELSD |
| Column | YMC PackPro C18, 3 μm, 12 nm, 150 × 4.6 |
| | mm, No. 041585193 (W) |

TABLE 3

| | |
|---|---|
| 0 minutes | 7% solvent B |
| 22.0 minutes | 19% solvent B |
| 29.0 minutes | 85% solvent B |
| 29.1 minutes | 90% solvent B |
| 31.0 minutes | 90% solvent B |
| 31.1 minutes | 7% solvent B |
| 36.0 minutes | 7% solvent B |

The results of the distribution of the various molecular weight procyanidins are illustrated in the HPLC chromatogram of FIG. 1. The horizontal axis represents the time frame under which the test was conducted, from the start at 0 minutes through 36 minutes. The vertical axis represents the absorption intensity at 275 nm. The narrow peaks at from 2.5 minutes through 24 minutes represents a full spectrum of phenolic compounds having approximate size respectively from monomers, dimers, trimers to low and mid-range oligomers, to <10mers or polymeric phenols. Highly significant, however, was the broad peak at from 26 minutes through 29 minutes, suggesting those phenolic compounds have sizes much large than 10mers or 3000 amu/3 kDa.

### EXAMPLE 3

In order to more accurately identify and select samples containing polymeric procyanidins having atomic mass units of >3000 amu to make more precise determinations of molecular weight ranges useful for producing compositions >35% total phenols comprising significant amounts of these >3000 amu polymer, Centrifugal Partition Chromatography (herein “CPC”) was then used to collect and combine sample fractions representing elution fractions over time. A CPC solvent system consisting of hexane-ethyl acetate-methanol-water (0.25:6:1:5, v/v) was utilized for fractionation of a cranberry extract characterized as having >35% total phenols, to produce samples for further analysis. To provide a pattern of elution, eluted material was collected in test tubes over time and the contents analyzed by analytical HPLC. Based on the composition of the test tubes, the contents were combined into eight fractions: characterized as five mobile phase fractions, which eluted first, and three stationary phase fractions, which eluted later, the largest polyphenols having been held by the columns most tightly.

More particularly, samples 1-4 were prepared by dissolving 9 grams of dried cranberry extract containing >35% total phenols into 40 ml of mobile phase and 50 mL of stationary phase. Sonication was utilized to thoroughly dissolve the material, which possessed a dark red color and did not appear viscous. The solution was filtered prior to being loaded into a rotor that was spinning at 1,500 RPM with a flow-rate of 20 mL/minute. The flow-rate was increased to 25 mL/minute after collecting into 20 test tubes. Thereafter, once 185 test tubes were collected, phase replacement was initiated at 250 RPM and 25 mL/minute. 26 mL were collected into each test tube; 240 test tubes were collected. HPLC analysis was performed with every third test tube (test tube #1-10), with every fifth test tube (test tube #15-30), with every eighth test tube (test tube #38-70), with every ninth test tube (test tube #79-187), and with every second test tube (test tube #197-237). 200 μl of the test tube samples were evaporated and then reconstituted with 500 μl of 20% aqueous methanol. The test tubes were combined into eight fractions and evaporated to dryness on a rotary evaporator. Fractions were transferred utilizing either acetone or methanol with a small volume of water.

Noticeable in the RP-HPLC data of the three stationary phase fractions, was a late eluting very large broad HPLC peak observed (˜27 minutes retention time), consistent with polymeric procyanidins. The stationary fractions were the most polar, most retained material and they constituted 51% of the starting cranberry extract by weight.

Samples 1-4 were obtained by CPC fractionation and were identified as Mobile #1, Mobile #2, Stationary #1 and Stationary #2 contained mostly polymeric procyanidins as determined by RP-HPLC and were selected for further analyses. Gel Permeation Chromatography was selected as an appropriate analytical method for determining the molecular weights of these oligomeric and polymeric phenolic components based on published details (J. Chromatog. A, 2006, 1112,112-20).

Samples 5 and 6 for GPC analysis were two samples of cranberry extract, one produced with SP-207 resin and another produced with Lewatit 1064 resin, generally according to the techniques described in U.S. Pat. No. 7,306,815, with both Samples 5 and 6 standardized to >30% total phenols as determined by Folin-C assay.

Samples 1-6, being based on cranberry extracts, contained many different polyphenols with a range of molecular weights, rather than containing a single phenolic compound with one molecular weight. Accordingly, Samples 1-6 were then analyzed using standardized Gel Permeation Chromatography (“GPC”) to determine the relative molecular weight distribution of the polyphenol extracts contained in each sample.

Standardized GPC calculates molecular weight values through a comparison between the retention times of samples of unknown molecular weight with those of standards of known molecular weight. This method assumes that if two molecules are of the same size in solution then they are the same molecular weight. The accuracy of this assumption is directly dependent upon the similarity between the hydrodynamic volume of the standards and that of the sample for a given molecular weight. Factors which are important in determining the hydrodynamic volume of a polymer includes the polarity and rigidity of a polymer, as well as the presence of charged functional groups incorporated into the polymer. It is therefore desired to keep the chemistry of the standards (charge, polarity, chain stiffness) as similar as possible to that of the samples.

In addition, it is assumed in standardized GPC that the only separation mechanism present is separation by size. Other factors such as sample-column or sample-sample interactions can result in non-sized based separation effects. Tetra-detection GPC can be used to confirm the absence of these effects and serves as a good way to validate a standardized GPC method. The accuracy of standardized GPC is generally expected to be within 10% of the accurate value when a purely sized-based separation is obtained and the hydrodynamic volumes of the standards correlate well with those for the sample. In comparison, tetra-detection GPC typically has accuracy on the order of 5% and makes no similar assumptions about sample hydrodynamic volume or separation by size.

When using GPC to describe the molecular weight distribution of a polymer numerically, three different molecular weight averages are commonly used. These are the number average molecular weight (Mn), the weight average molecular weight (Mw), the peak molecular weight (Mp), and the Z average molecular weight (Mz). Mn provides information about the lowest molecular weight portion of the sample. Mw is the average in the sample closest to the center of the curve and Mz represents the highest molecular weight portion of the sample.

Prior to testing using standard GPC methods, Samples 1-6 were dissolved overnight in dimethyl sulfoxide (DMSO) with 0.1 M lithium bromide at room temperature with gentle agitation, yielding dark red/purple solutions. No insoluble material was observed; therefore, the samples were injected without further preparation. Molecular weight distribution data is summarized in FIG. 2 which contains the molecular weight distribution curves containing individual traces of each of samples 1-6. The average molecular weights Mn, Mw, Mp, and Mz for each of the six samples are also indicated on the graph for ready comparison.

Results of the GPC testing are further summarized below in Table 4, with data reported for each value being the average of two runs.

TABLE 4
Average Molecular Weight (relative to polysaccharide standards)

| Sample No. | Mₙ | M_{w} | M_{z} | M_{w}/Mₙ |
|---|---|---|---|---|
| 1 | 130 | 450 | 1131 | 3.48 |
| 2 | 186 | 537 | 1279 | 2.88 |
| 3 | 566 | 2260 | 5037 | 3.99 |
| 4 | 949 | 3154 | 5906 | 3.32 |
| 5 | 349 | 1889 | 5188 | 5.41 |
| 6 | 404 | 3268 | 12695 | 8.09 |

The samples all exhibited multimodal low molecular weight peaks with M_{z} values ranging from 1 kDa to approximately 12 kDa relative to polysaccharide standards. However, Samples 1 and 2, which represent the first two mobile phase fractions collected during CDC separation contain the smallest and most polar impurities in the cranberry extracts containing >35% total phenols. These smallest and most polar impurities include organic acids and sugars. More specifically, Sample 1 (the first eluting mobile phase) likely included mostly organic acids commonly found in cranberry extracts, namely malic acid (134 amu), citric and quinic acids (192 amu) which are included in the Mn value of 130. Sample 2 (the second eluting mobile phase) likely included mostly cranberry sugars, including fructose and glucose (180 amu) and sucrose (342 amu), which are included in the Mn value of 186.

Taken as a whole, the data summarized in Table 4 confirms the presence of smaller monomers and oligomeric procyanidins in both the CPC mobile phase fractions, denoted as Samples 1 and 2, (M_{z} averages 1131 amu, 1279 amu), as well as the presence of large polymeric procyanidins in both the CPC stationary fraction, denoted as Samples 3 and 4 (M_{z}˜5-6 kDa). Samples 1-6 all exhibited multimodal molecular weight peaks with M_{z} values ranging from ˜1 kDa to 12 kDa, an M_{z} value much greater than the other samples disclosed in the literature.

Samples 5 and 6 of cranberry extract produced by Lewatit 1064 and SP207 resins, respectively, both contained very high molecular weight procyanidin polymers of M_{z} averages 5 kDa and 12 kDa, respectively, showing that the much higher molecular weight polymeric phenolic components are able to be captured and released by these two resins, which are normally lost in most purification processes. Also important was the capture and release by these two resins of the lower molecular weight phenolic components (i.e., monomers, dimers, trimers, oligomers) as well, providing a pathway to producing a wide spectrum phenolic extract containing as much as possible of all the naturally present phenolics in the cranberry raw material used.

The polydispersity index is a way to quantify the broadness of a molecular weight distribution of a polymer, and is defined by:

$$Polydisperity Index = \frac{{M}_{w}}{{M}_{N}}$$

The larger the polydispersity index, the broader the molecular weight of the components tested. It is clear the types of components are a complex mixture from the polydispersity index of 2.88-3.38 in the first four samples, which are mobile and stationary elution fractions from CPC. The highest polydispersity index in Table 4 is from the two samples of standardized cranberry extracts produced from various resins, samples 5 and 6. Also, the M_{z} higher averages in Table 4 are due to enhanced sensitivity to the presence of very high molecular weight polymers in samples 3-6. Herein, all subsequent molecular weights mentioned in kDaltons are based on their GPC Mz values as described herein.

### EXAMPLE 4

Because there had been differences of opinion amongst those skilled in the art regarding the efficacy of cranberry concentrates as bacterial anti-adhesion agents (herein “AAA”) preventing urinary tract infections (herein “UTIs”), tests were undertaken to assess the efficacy of various phenolic extracts manufactured according to one column elution processes using either SP-207 or Lewatit 1064 resin. Samples were suspended at a concentration of 60 mg/ml in a phosphate buffered saline solution neutralized with 1 N NaOH, diluted serially, and tested for bacterial anti-adhesion activity utilizing a human red blood cell (herein “HRBC”) hemagglutination assay specific for uropathogenic P-fimbriated E. coli according to Foo et al. (Phytochemistry, 2000). The concentration at which hemagglutination activity was suppressed by 50% was recorded as an indicator of the strength of the bacterial anti-adhesion activity. Anti-adhesion assays were repeated three times and the results averaged. Controls were utilized. The final concentration at which anti-adhesion activity could be detected was recorded, with the smaller the number, the greater the anti-adhesion activity. Results of the tests are summarized below in Table 5.

TABLE 5

| | | AAA | AAA |
|---|---|---|---|
| | | Whole | Whole |
| | | Product | Product |
| | | (mg/mL) | (mg/mL) |
| No. | Sample Description | [symbol] P-type | Type 1 |
| 1 | Cranberry concentrate from SP-207 | 3.75 | ** |
| | column standardized to 30% total | | |
| | phenols | | |
| 2 | Cranberry concentrate from SP-207 | 0.94 | 7.5 |
| | column standardized to 30% total | | |
| | phenols | | |
| 3 | Cranberry concentrate from SP-207 | 0.23 | 1.87 |
| | column standardized to 30% total | | |
| | phenols | | |
| 4 | Cranberry concentrate from Lewatit | 0.47 | 7.5 |
| | column standardized to 30% total | | |
| | phenols | | |
| 5 | Cranberry concentrate from Lewatit | 0.23 | 0.94 |
| | column standardized to 30% total | | |
| | phenols | | |
| 6 | Cranberry concentrate from Lewatit | 0.47-0.23 | 0.94 |
| | column standardized to 30% total | | |
| | phenols | | |
| 7 | Cranberry concentrate from Lewatit | 0.23 | 7.5 |
| | column standardized to 30% total | | |
| | phenols | | |
| 8 | Blueberry concentrate from Lewatit | 0.94 | 15 |
| | column standardized to >35% total | | |
| | phenols | | |
| 9 | Cranberry concentrate from Lewatit | 0.12-0.06 | 0.94 |
| | column standardized to >35% total | | |
| | phenols | | |
| 10 | Cranberry concentrate from Lewatit | 0.23 | 3.75 |
| | column standardized to >35% total | | |
| | phenols | | |
| 11 | Cranberry concentrate from Lewatit | 0.23 | 7.5 |
| | column standardized to >35% total | | |
| | phenols | | |

Because the P-type assay is the most sensitive, the values obtained were considered the most meaningful. Sample No. 9 had the lowest values in both the P-type and Type 1 assays and therefore the highest anti-adhesion activity overall and considered to be excellent. In contrast, sample No. 1 had the least anti-adhesion activity in both assays. The Type 1 test results were not given as much weight, as it is not as sensitive as the P-type tests, in part because fructose and some other saccharides can give a positive result without necessarily having in vivo activity.

### EXAMPLE 5

Additional tests were then undertaken with different fruit extract to try to identify, which phenolic qualities, be it degree of polarity, degree of polymerization, molecular weight, etc., were the most active with respect to bacterial anti-adhesion activity. In these tests, samples were suspended in a 250 μL phosphate buffered saline solution neutralized with 1 N NaOH, diluted serially, and tested for bacterial anti-adhesion activity utilizing a HRBC hemagglutination assay specific for uropathogenic P-fimbriated E. coli. Again, the concentration at which hemagglutination activity was suppressed by 50% was recorded as an indicator of the strength of the bacterial anti-adhesion activity. Anti-adhesion assays were repeated three times and the results averaged. Controls were utilized. The final concentration at which anti-adhesion activity could be detected was recorded, with the smaller the number, the greater the anti-adhesion activity. Results of the tests are summarized below in Table 6, with the qualitative sample descriptions for the CPC fractions of Sample Nos. 7-25 based on HPLC retention time data only.

TABLE 6

| | | AAA |
|---|---|---|
| | | (mg/mL) |
| No. | Sample Description | P-type |
| 1 | Elderberry extract >35% total phenols eluted from SP- | negative |
| | 207, prior to CPC fractionation | |
| 2 | Blueberry extract >35% total phenols eluted from SP- | 6.61 |
| | 207, prior to CPC fractionation | |
| 3 | Cranberry extract >35% total phenols eluted from SP- | 0.78 |
| | 207, prior to CPC fractionation | |
| 4 | Elderberry extract >35% total phenols eluted from | negative |
| | Lewatit 1064, prior to CPC fractionation | |
| 5 | Blueberry extract >35% total phenols eluted from | 2.73 |
| | Lewatit 1064, prior to CPC fractionation | |
| 6 | Cranberry extract >35% total phenols eluted from | 1.52 |
| | Lewatit 1064, prior to CPC fractionation | |
| 7 | Elderberry extract >35% total phenols eluted from SP- | negative |
| | 207 followed by collection of CPC fraction containing | |
| | substantial amounts of the least polar phenolic | |
| | compounds including monomers and dimers | |
| 8 | Elderberry extract >35% total phenols eluted from SP- | negative |
| | 207 followed by collection of CPC fraction containing | |
| | substantial amounts of slightly more polar phenolic | |
| | compounds including monomers, dimers and smaller | |
| | oligomers | |
| 9 | Elderberry extract >35% total phenols eluted from SP- | negative |
| | 207 followed by collection of CPC fraction containing | |
| | substantial amounts of more polar phenolic compounds | |
| | including mostly smaller oligomers <10mers | |
| 10 | Elderberry extract >35% total phenols eluted from SP- | negative |
| | 207 followed by collection of CPC fraction containing | |
| | yet more polar phenolic compounds including | |
| | oligomers through 10mers | |
| 11 | Elderberry extract >35% total phenols eluted from SP- | 5.6 |
| | 207 followed by collection of CPC fraction containing | |
| | substantial amounts of highly polar phenolic | |
| | compounds >10mers and some anthocyanins | |
| 12 | Elderberry extract >35% total phenols eluted from SP- | negative |
| | 207 followed by collection of CPC fraction containing | |
| | the most polar phenolic compounds comprising mostly | |
| | anthocyanins | |
| 13 | Blueberry extract >35% total phenols eluted from | negative |
| | SP-207 followed by collection of CPC fraction | |
| | containing substantial amounts of the least polar | |
| | phenolic compounds including monomers and dimers | |
| 14 | Blueberry extract >35% total phenols eluted from | negative |
| | SP-207 followed by collection of CPC fraction | |
| | containing substantial amounts of the slightly | |
| | more polar phenolic compounds including monomers, | |
| | dimers and less polar oligomers | |
| 15 | Blueberry extract >35% total phenols eluted from | negative |
| | SP-207 followed by collection of CPC fraction | |
| | containing substantial amounts of the polar | |
| | phenolic compounds including oligomers | |
| 16 | Blueberry extract >35% total phenols eluted from | negative |
| | SP-207 followed by collection of CPC fraction | |
| | containing substantial amounts of the more polar | |
| | phenolic compounds including oligomers <10mers | |
| 17 | Blueberry extract >35% total phenols eluted from | 25.2 |
| | SP-207 followed by collection of CPC fraction | |
| | containing substantial amounts of the very polar | |
| | phenolic compounds >10mers including anthocyanins | |
| 18 | Blueberry >35% total phenols eluted from SP-207 | negative |
| | followed by collection of CPC fraction containing | |
| | substantial amounts of the most polar phenolic | |
| | compound which are mostly anthocyanins | |
| 19 | Cranberry extract >35% total phenols eluted from | 3.14 |
| | SP-207 followed by collection of CPC fraction | |
| | containing substantial amounts of the least polar | |
| | phenolic compounds including monomers and dimers | |
| 20 | Cranberry phenolic less polar fraction, including | 3.36 |
| | monomers, dimers and trimers. | |
| 21 | Cranberry phenolic mid-polarity fraction, including | 27.36 |
| | dimers, trimers, tetramers. | |
| 22 | Cranberry phenolic slightly more polar fractions | 12.13 |
| | including lower oligomers (trimers and higher | |
| | oligomers) | |
| 23 | Cranberry phenolic mid-polar fraction including | negative |
| | <10mers | |
| 24 | Cranberry phenolic polar fraction <10mers | negative |
| 25 | Cranberry highest molecular weight polyphenols | 0.37 |
| | mostly >10mers plus some anthocyanins | |

In considering the data, certain patterns were observed. Firstly, sample fractions from cranberry and blueberry extracts containing >35% total phenols were quite active in the assay, whether produced using PS-DVD crosslinked porous gel packed columns containing SP207 or Lewatit 1064 resins. However cranberry extracts produced using either SP207 or Lewatit 1064 resins were significantly more active than comparable blueberry extracts and comparable elderberry extracts not active at all in the assay.

Regarding specific fractions, one elderberry extract fraction tested and one blueberry medium polarity phenolic fraction tested were both active, and they each contained larger oligomers/polymers and some anthocyanins. Most cranberry phenolic fractions tested were active, whether they were fractions containing non-polar phenolic components such as monomers, dimers and trimers, or fractions containing the more polar, higher oligomers. Only one cranberry phenolic fraction of medium polarity was inactive. Single fractions of blueberry and elderberry each containing both the higher oligomer/polymer and anthocyanins were less active, with all their other fractions inactive in the assay.

The most active sample tested was also the most polar sample of cranberry polyphenols, containing anthocyanins and larger procyanidin polymers. This cranberry sample was 2-5 times more active than the cranberry total phenol resin extracts. The top three most active fractions in the P-type assay for UTI are one cranberry fraction and two resin extracts containing high amounts of larger molecular weight polymers. The cranberry fractions containing much lower molecular weight species (i.e., monomers, dimers and trimers and lower oligomers) were also active in the assay to a lesser degree.

The data support the potency for the cranberry extracts for UTI prevention and treatment, particularly in mammals, is optimized by increasing the amount of total phenols, including both the higher molecular weight polyphenols and the anthocyanins in the extract produced. The data also support the novel use of blueberry extracts containing >35% total phenols for either prevention or treatment of UTI, or other urogenital conditions, as well, either alone or in combination with cranberry as both are quite active in the P-type-assay. Since one fraction from elderberry was also active, and that fraction contained larger (Example 1 RP-HPLC data) polyphenols as well as anthocyanins, it is concluded that elderberry total phenol enriched extracts with higher molecular weight polyphenols are expected to be useful for either prevention or treatment of UTI, or other urogenital conditions, either alone or in combination with cranberry and/or blueberry as both are quite active in the P-type-assay.

In an aspect of the present disclosure, the enriched, full-spectrum, polyphenolic extract may contain at least 1% anthocyanins. It is also contemplated that at least 5% w/w of the polyphenolic extract may be greater than 4.5 kDalton. It is further contemplated that at least 5% w/w of the polyphenolic extract may be greater than 7.5 kDalton.

### EXAMPLE 6

The spike protein, which is located on the outside of a coronavirus, enables Severe Acute Respiratory Symptoms (“SARS”) Covid2 (“Cov2”) virus to enter human cells. It has been shown that Covid binds to human angiotensin-converting enzyme 2 (herein “ACE2”), a monomeric transmembrane protein present on many types of human cells. Since the spike protein governs viral attachment and virus-cell membrane fusion, which subsequently determines the fate of viral replication and infectivity, it has therefore been of interest as a therapeutic target and was selected by the inventors here to examine phenolic extracts potential in vitro bioactivity in an ACE2 assay, for utility as prevention and treatment of COVID infections. Indeed, in U.S. Pat. No. 7,306,815 it had been shown that a polyphenol extract of elderberry can have modest in vitro activity against a coronavirus. Accordingly, ACE2 testing of cranberry, blueberry and elderberry phenolic-enriched extracts and their fractions was undertaken and wide-ranging activity was found.

Initially, a study of the inhibition of ACE2 binding of COVID spike protein by polyphenol extracts, papain, and nicotine was studied. Polyphenol extracts tested included Cranberry, Blueberry and Elderberry. In this study, high binding polystyrene microplates were coated with SARS Cov2 spike protein (0.5 μg/ml) overnight. Plates were then washed with phosphate buffer then blocked at 37° C. using 2% (weight/volume (herein “w/v”) bovine serum albumin (BSA) for 2 hours. All dilutions were performed in 0.2% (w/v) bovine serum albumin. Plates were then washed and human biotinylated ACE2 was added. Test substances or standards were added and plates were incubated for 1 hour. Treatment concentrations for polyphenol mixtures ranged from 7.8 to 125 μg/ml. Two positive controls (papain and nicotine) were used to validate the ACE assay. Treatment concentrations for Papain ranged from 6.1 to 100 μg/ml. Treatment concentrations for nicotine ranged from 7.8 to 500 ng/ml. Plates were then washed and HRP-Streptavidin, available from Sigma-Aldrich, was added. Plates were then incubated for 1 hour in the dark, and then washed. Thermo Quanta-Blue fluorescent substrate, available from Thermo-Fisher Scientific, was added and plates were allowed to react for 1 hour at room temperature in the dark. Plates were then read (ed325/em420) on a plate reader. Results reported represented negative control subtracted data as a percentage of the positive control (also negative control subtracted).

Relative Binding (% Positive control) SARS Cov2 Spike protein ACE2 Binding inhibition by polyphenols enriched extracts of Blueberry, Cranberry and Elderberry was measured. Blueberry and, to a lesser extent, elderberry polyphenol extracts showed a concentration dependent reduction in the binding of ACE2 to SARS Cov2 spike protein.

In all cases there was 20-30% reduction in ACE2 binding at 125 mg/ml phenol-enriched extracts. The lack of an apparent concentration dependent reduction in binding in the case of cranberry and mostly flat curves (in all cases) most likely the result of the highly enriched polyphenol extracts inhibition mechanism being saturable, due to interactions with the matrix such as the BSA (which is well known for polyphenols).

To explore this preliminary ACE2 in vitro bioactivity more closely, CPC fractions derived from Cranberry, Blueberry, and Elderberry extracts (as described earlier in Example 6) in which the least polar fractions containing monomers were eluted first and the most polar fractions containing both anthocyanins and proanthocyanins >10mers were collected last, were then tested in an ACE2 bioassay. Fractions tested not only included fractions highly enriched in various polyphenols of different sizes and polarity, but also included fractions comprising mainly sugars and acids. Of 45 fractions tested, the most active sets of ACE2 samples are summarized below in Table 7, ranked according to the percentage of inhibition of ACE2 activity as compared to the most active sample tested.

TABLE 7

| | | % Inhibition |
|---|---|---|
| Sample | | vs. |
| No. | Sample Description (all @ 50 μg/ml) | Sample No. 1 |
| 1 | Blueberry fraction 4 smaller oligomers | 100 |
| 2 | Blueberry fraction 6 oligomers | 94.6 |
| 3 | Cranberry fraction 8 >10 mers + anthocyanins) | 89.5 |
| 4 | Cranberry fraction 9 >10mers + anthocyanins) | 84.1 |
| 5 | Elderberry fraction 4 smaller oligomers | 83.9 |
| 6 | Blueberry fraction 1 monomers, dimers, trimers | 78.8 |
| 7 | Blueberry fraction 7 >10 mers + anthocyanins) | 77.1 |
| 8 | Blueberry fraction 2 monomers, dimers, | 76.1 |
| | trimers, tetramers | |
| 9 | Cranberry-Lewitit >35% total phenols* | 72.3 |

The most active fractions (>75% inhibition) are listed in Table 7, based on observed potency by concentration differences, and Samples 1-8 are greater than 75% inhibition by concentration at 50 μg/ml. These are cranberry fractions 8 and 9. blueberry fractions 1, 2, 4 and 6, and elderberry fraction 4.

The data suggest that a wide range of phenolic components are bioactive in the ACE2 assay, ranging from monomers to >10mers by HPLC. The data also suggest that phenolic components from a number of sources, including cranberry, blueberry and elderberry, are bioactive in the ACE2 assay and that enriching those components would provide uses in the prevention and treatment of COVID infections thru this mechanism of action. Also noted was that blueberry-derived phenolic enriched extracts and fractions had the widest range of activity and an excellent source for ACE2 active phenolic compounds. Overall, the bioactive components highlight that fractions containing higher molecular weight polyphenolics are active and all other active samples are fractions with smaller phenolics (ranging from monomers-oligomers <10mers: Blueberry fractions 1, 2 and 4, Elderberry fraction 4) with sizes based on RP-HPLC retention times (“Rt's”) as well as the relative polarities from the CPC elution order.

However, positive ACE2 assay activity has broader relevance beyond COVID specifically and viruses more generally. Indeed, ACE2 is also implicated in cardiovascular, lung and kidney diseases, diabetes and pregnancy complications. See, e.g., I. Hamming, et al., “The emerging role of ACE2 in physiology and disease”, J. Pathol., 2007: 212: 1-11. While the use of super-enriched phenolic extracts in prevention and/or treatment of these disorders is now proposed, development of a more efficient route of oral delivery to increase bioavailability and absorption is needed and studies utilizing two models were carried out. Experiments were conducted with respect to increasing bioavailability of phenolic compounds across intestinal walls using the well stablished CaCo2 model. Experiments were conducted utilizing the also well-established MDCK model to evaluate a more efficient route of delivery across the blood-brain barrier, as are described below.

### EXAMPLE 7

Historically, most commercial methods for purification and concentration of phenolic compounds for oral consumption were developed to optimize purification of single classes of polyphenols, and these methods were usually focused on the smaller molecular weight components a particular class of phenols. Moreover, it has not been fully appreciated that commercially available extracts enriched so as to concentrate phenols from fruits and vegetables to produce a product constituting >10% total phenols, thereby allowing individuals to obtain the benefits of phenolic compounds without necessitating the consumption of large volumes of fruit or vegetable juices. Such extracts do not contain a full spectrum of phenolic compounds from monomers to the phenols having the largest degrees of polymerization. The problem is exacerbated by the fact that the amount and distribution of antioxidant phenolic components vary substantially among different fruits, even among different cultivars of the same fruits. Furthermore, both biotic and abiotic factors such as genetics, climate, water availability, and crop management have an important role in the level of bioactive compounds and overall antioxidant capacity in the raw materials.

For all these reasons, products commercially available do not contain the widest range of molecular sizes, classes and polarities, and having observed that there is significant bioactivity for both small and larger molecular weight polyphenol components in the AAA bioassays (Example 4) and ACE2 bioassays (Example 6), a separation method was developed by the inventors hereof to maximize the purification across these different sized and polarity ranges of polyphenols, and to produce an extract enriched in all these classes of polyphenols in extracts >35% total phenols.

Generally, the extraction and purification method of the present disclosure, starting from plant material such as raw fruits, vegetables, frozen fruit juice concentrate, or other plant materials, begins with extraction with water and/or a water miscible solvent, and then filtration of a juice comprising the plant material. The preferred solvents are alcohol of 1-3 carbon atoms or acetone. The aqueous extract is used directly or after solvent removal. If the solvent is removed, the solid residue is re-dissolved in a solvent, preferably a lower alcohol or acetone, and filtered to create a raw extract. The raw extract is then diluted using acidified water to the approximate phenolic level of the initial plant material used. This creates “juice strength extract.” The soluble portion is then subjected to either a single column as described previously, or two columns, in series, of gel-permeation chromatography with, for example, divinyl benzene cross-linked gels such as Sepabeads™ SP207, a product of Mitsubishi Chemical Corporation, whose matrix is a modified styrene-DVB structure, see, http://www.diaion.com/en, or with Lewatit® VP OC 1064 MD PH, an adsorption resin in the form of white, opaque, porous beads, which comprises a crosslinked polystyrene matrix with a very high surface are but without any function groups, see, Sigma-Aldrich.com, or the like, using water or water and a water miscible solvent, with or without a buffer, as the mobile phase. The water miscible solvent is preferably a 1-3 carbon alcohol, acetone or acetonitrile. This causes the phenolic constituents in the extract to adhere to the column and non-phenolic contaminants to pass through the gel-packed column. The phenolic constituents remaining in the column are then washed with at least 3 to 25 column volumes of acidified water to remove sugars, organic acids, and any remaining non-phenolic contaminants.

A portion of the phenolic constituents are then eluted and collected using lower alcohol, acidified water in 50-90% to create a semi-purified polyphenolic extract. This semi-purified extract is then concentrated by removing ethanol and water. The concentrated product is then diluted with acidified water to create a semi-purified eluent.

The semi-purified eluent may then be loaded into a second column, again causing the phenolic constituents to adhere to the column and remaining non-phenolic contaminants to pass through the column. The phenolic constituents are then washed with at least 3 to 25 column volumes of acidified water. This wash again helps to remove sugars, organic acids, and non-phenolic contaminants adsorbed in the phenolic constituents.

The phenolic constituents are then eluted using acidified alcohols containing 1-3 carbons and collected to create an enriched polyphenolic eluent. The enriched polyphenolic eluent is then concentrated by removing the liquid therefrom. Additional polyphenolic constituents are then eluted using acidified alcohols containing 1-3 carbons and collected creating an enriched polyphenolic eluent. The enriched polyphenolic eluent is then concentrated by removing the liquid. The result is an enriched, full-spectrum, polyphenolic extract which contains greater than 35% by weight total phenols and at least 10% by weight of the phenolic extracts are greater than 2.5 kDaltons. The result may be enriched, full-spectrum, polyphenolic extract which contains 35%-95% by weight total phenols. The enriched, full-spectrum, polyphenolic extract may be combined with trisodium citrate to produce an oral composition exhibiting enhanced bioavailability. In the alternative to trisodium citrate, the enriched, full spectrum, polyphenolic extract may be combined with cyclodextrin to produce and oral composition exhibiting enhanced bioavailability.

This method is described below, with the individual quantities and volumes to be used as can be determined by one of skill in the art to which the present disclosure pertains:
Step 1. Plant materials, such as raw fruits, vegetables, frozen fruit juice concentrate, or other plant materials are used to obtain a raw extract.
Step 2. The raw extract is diluted with acidified water such as water acidified with sulfuric acid, acetic acid or hydrochloric acid to the approximate phenolic strength of the plant material used in the extraction, a juice strength extract, i.e., 5-6 times diluted from concentrate, with filtration if needed.
Step 3. The product obtained from step 2 is loaded onto a PS-DVD crosslinked porous gel packed column such as a Mitsubishi SP-207 resin, Lewatit OC 1064 resin, or the like, or a combination of such resins
Step 4. The product adhering to the column in step 3 is washed with acidified water to remove sugars and organic acids with from 3-25 column volumes of acidified water.
Step 5. The polyphenols adhering to the column are acidified with lower alcohol/acidified water in 50-90% and eluted from the gel column.
Step 6. The ethanol and water are concentrated, preferably using lyophilization or vacuum drying, and then diluted with acidified water to approximate volume in step 2.
Step 7. The eluent is then loaded onto a first column containing first resin comprising a PS-DVD crosslinked porous gel packed such as a Mitsubishi SP-207 resin, Lewatit OC 1064 resin, or the like, or a combination of such resins.
Step 8. The column is then washed with at least 3-10 column volumes of acidified water.
Step 9. The polyphenols are then eluted with acidified lower alcohols (1-3 carbons) from the gel column.
Step 10. This later eluting fraction is then concentrated using lyophilization, vacuum drying, spray drying or an equivalent method.
Step 11. Steps 7-10 are then optionally repeated with utilizing a second column containing a second resin which is also a PS-DVD crosslinked porous gel packed such as a Mitsubishi SP-207 resin, Lewatit OC 1064 resin, or the like, or a combination of such resins, to produce a super-enriched extract containing >35% total phenols comprising a full spectrum of phenolic components.
Step 12. Optionally, the weight % of total phenols in the resulting dry mixture can then be determined by Folin-C.
Step 13. Also optionally, the weight % of total phenols in the resulting dry mixture which is above 3000 Daltons/3 kDalton can be determined using GPC, HPLC, capillary zone electrophoresis (CZE) or various Mass Spectrometry(MS) techniques.

The phrase “full spectrum of phenolic components” as used herein which make up the polyphenolic extracts of the present disclosure, is defined to include the following sizes: monomers, dimers, trimers, oligomers of from 1.2 kDalton through 3 kDalton, and polymers of >2.5 kDalton, where the polymers constitute at least 10% by weight of the polyphenolic extract and the polyphenolic extract contains only native polyphenols, i.e., only those polyphenol components which are naturally-occurring metabolites from the specific plant material which the extract is concentrated and derived. The term “phenolic components” is understood to include different combinations of classes of polyphenols, including but not limited to anthocyanins, proanthocyanins, proanthocyanidins, procyanins, procyanidins, phenolic acids, flavonoids, as well as non-hydrolyzable and hydrolysable tannins.

The most preferred plant materials to be used in the above method are the so-called “red and purple fruits” including but not limited to blueberries, bilberries, elderberries, plums, blackberries, marionberries, strawberries, red currants, black currants, cranberries, cherries, red raspberries, black raspberries, grapes and currants. Other fruits, vegetables and edible plant material may also be used, including but not limited to hibiscus flowers, bell peppers, beans, peas, red cabbage, purple corn, violet sweet potatoes, olives, pomegranates, mangosteen, apples, hawthorn, gooseberries, acai, and oranges. Combinations of different plant material may also be used. Plant materials are also not limited to whole fruits and vegetables but may also include skins, peels, fruits, nuts, hulls or seeds thereof. Plant sources containing <0.5% total phenols dry weight are excluded.

Alternatively, plant material may be obtained from a Vaccinium species, for example, Vaccinium macrocarpon or Vaccinium angustifolium. Plant material may be obtained from a Sambucus species, for example, Sambucus nigra.

### EXAMPLE 8

The permeability of blueberry and elderberry polyphenols across the blood-brain barrier in the presence and absence of trisodium citrate was tested through a well-known MDCK model blood-brain barrier method. Approximately 1 mg/ml of either blueberry or elderberry extract in 0.1 M Dulbecco's phosphate buffered solution (herein “DPBS”) alone or in the presence of 10 mM trisodium citrate was added to a donor chamber of 3 side-by-side diffusion cells, with a confluent MDCK monolayer between (TEER ˜500 Ohm*cm2). Samples were taken at 15 minute intervals for 90 minutes and replaced with fresh isotonic DPB S. Samples were taken from the donor chambers at time 0 and at 90 minutes. Samples were then analyzed using a modified Folin-C method and measured for total phenolic content. The results are summarized in FIGS. 3 and 4.

The permeability of blueberry extracts though a MDCK monolayer in the presence and absence of 10 mM trisodium citrate is illustrated by tracings 1 and 2 in the graph of FIG. 3. The vertical axis represents cumulative polyphenol permeation measured in μg propylgalate equivalents, while the horizontal axis represents time in minutes. Measuring at 15 minutes, cumulative polyphenol permeation for both Tracing 1 (blueberry extract only) and Tracing 2 (blueberry plus trisodium citrate) was reported as approximated 2.5 μg. Thereafter at each 15 minute interval measurably more blueberry extract in the presence of trisodium citrate passed through the MDCK monolayer, until at 90 minutes, approximately 7.0 μg cumulative polyphenol permeation for blueberry extract only was reported, while at 90 minutes over twice as much (15.0 μg) cumulative polyphenol permeation for blueberry extract in the presence of trisodium citrate was reported.

The permeability of elderberry extracts though a MDCK monolayer in the presence and absence of 10 mM trisodium citrate is illustrated by tracings 1 and 2 in the graph of FIG. 4. The vertical axis represents cumulative polyphenol permeation measured in μg propylgalate equivalents, while the horizontal axis represents time in minutes. Measuring at 15 minutes, cumulative polyphenol permeation for both Tracing 1 (elderberry extract only) and Tracing 2 (elderberry plus trisodium citrate) was reported as approximated 8 μg. Thereafter at each 15 minute interval measurably more elderberry extract in the presence of trisodium citrate passed through the MDCK monolayer, until at 90 minutes, approximately 27 μg cumulative polyphenol permeation for elderberry extract only was reported, while at 90 minutes substantially more 42 μg cumulative polyphenol permeation for elderberry extract in the presence of trisodium citrate was reported (>1.5×).

### EXAMPLE 9

The utility of using either cyclodextrins or sodium citrate as agents to enhance drug delivery of polyphenols and thereby improve the efficiency of polyphenols crossing the gut, as well as the blood brain barrier were also evaluated using various cellular models. More specifically highly enriched polyphenol extracts of both blueberry and elderberry have been tested and data for the enhancement of the diffusion enhancement across the intestinal barrier using the CaCo2 cell model are illustrated in FIGS. 5, 6 and 7.

In one experiment, four test solutions were prepared, each comprising 3 mL of blueberry polyphenol solution containing approximately 1 mg/ml in 0.1M DPBS solution which was then tested alone (No. 1) or in the presence of 10 mM alpha (α) cyclodextrin (No. 2), beta (β) cyclodextrin (No. 3), or gamma (γ) cyclodextrin (No. 4). Each sample was added to the donor chamber of each of 3 side by side diffusion cells with a confluent Caco2 monolayer between (TEER ˜900 Ohm*cm2). Samples were taken at 15 minute intervals for 90 minutes and replaced with fresh isotonic DPBS. Samples were also taken from donor chamber at time 0 and at 90 minutes. Samples were then analyzed using modifications of the Folin-C method for total phenolic content.

The results for these studies are summarized graphically in FIG. 5, with the vertical axis representing cumulative polyphenol diffusion in μg propylgalate equivalents, and the horizontal axis representing time in minutes when samples were measured. Trace No. 1 represents polyphenol permeation through the membrane barrier of the blueberry extract sample No. 1, in which no cyclodextrin was present, in which permeation was measured at 10 μg/ml after 15 minutes and had increased to 20 μg/ml at 90 minutes. The permeation measured for blueberry extract with α-cyclodextrin (Trace No. 2) and for blueberry extract with γ-cyclodextrin (Trace No. 4) did not show any sustained improvement over the permeation measured with blueberry extract alone. However, polyphenol permeation through the membrane barrier of the blueberry extract sample in the presence of β-cyclodextrin (Trace No. 3) shows marked improved permeation as compared to the three other compositions tested. Permeation for blueberry extract in the presence of β-cyclodextrin was consistently better at all times measured at 30 minutes through 90 minutes, with a final measurement of 25 μg/ml at 90 minutes.

In another experiment, two test solutions were prepared, each comprising 3 mL of a 30% elderberry polyphenol solution containing approximately 1 mg/ml in 0.1M DPBS solution which was then tested alone (No. 1) and in the presence of 10 mM trisodium citrate (No. 2). Each sample was added to the donor chamber of each of 3 side by side diffusion cells with a confluent Caco2 monolayer between (TEER ˜900 Ohm*cm2). Samples were taken at 15 minute intervals for 90 minutes and replaced with fresh isotonic DPB S. Samples were also taken from donor chamber at time 0 and at 90 minutes. Samples were then analyzed using modifications of the Folin-C method for total phenolic content.

The results for this study are summarized graphically in FIG. 6, with the vertical axis representing cumulative polyphenol diffusion in mg propylgalate equivalents, and the horizontal axis representing time in minutes. Trace No. 1 represents polyphenol permeation through the membrane barrier of the elderberry extract, sample No. 1, in which no trisodium citrate was present, in which cumulative permeation 0.04 mg at 90 minutes. The cumulative permeation measured for elderberry extract in the presence of trisodium citrate (Trace No. 2) show slight improvement over the permeation measured with blueberry extract alone, at 0.05 mg at 90 minutes.

Using the same CaCo2 methodology as just described for the elderberry experiments, a blueberry and a cranberry extract, each alone and in the presence of trisodium citrate, were tested for membrane permeation by constituent polyphenols. Results are for these studies are summarized in FIG. 7, with the vertical axis representing cumulative polyphenol diffusion in μg propylgalate equivalents, and the horizontal axis representing time in minutes when samples were measured. Trace No. 1 represents polyphenol permeation through the membrane barrier of the blueberry extract in which no trisodium citrate was present, with permeation was measured at approximately 7.5 μg at 90 minutes. In contrast, as represented by Trace 2, the sample containing blueberry extract in the presence of trisodium citrate, cumulative polyphenol penetration increased to 14 μg at 90 min. Trace No. 3, which represents polyphenol permeation through the membrane barrier of the cranberry extract in which no trisodium citrate was present, indicates that cumulative permeation was measured at approximately 4 μg at 90 minutes, while as represented by Trace 4, the sample containing cranberry extract in the presence of trisodium citrate, cumulative polyphenol penetration increased to approximately 5 μg at 90 min.

The utility of using trisodium citrate as a delivery agent to increase the efficiency and efficacy of polyphenols crossing both the gut as well as the blood brain barrier has been evaluated using various cellular models for both, e.g., CaCo2 and MDCK respectively. Enriched polyphenol extracts of cranberry, blueberry and elderberry have been tested in both models and data for the enhancement of the diffusion enhancement across the intestinal barrier using the CaCo2 cell model as well as crossing the blood brain barrier are described above. The utility of cyclodextrin with enriched blueberry extract has been also tested in the CaCo2 model and found to increase absorption. In summary, the addition of trisodium citrate with berry extracts have been shown to significantly enhance bioavailability in both the MDCK and CaCo2 models. Furthermore, elderberry extract with trisodium citrate gave a statistically significant increase in permeability using the MDCK monolayers model.

In view of the above, in the formulations and methods of the present disclosures, a molar ratio of trisodium citrate to polyphenols of 0.3:1 is one viable formulation. Broadly speaking, however, a range of acceptable molar ratios for use in the present disclosures of trisodium citrate to polyphenols of from 0.3:1 to 1:200 would be an acceptable formulation. A preferred range of molar ratios trisodium citrate to polyphenols of 1:10 to 1:20 would be an acceptable formulation. A most preferred molar ratio range of trisodium citrate to polyphenols of 1:12 to 1:16 would be an acceptable formulation.

In view of the above, in the formulations and methods of the present disclosures, a molar ration of 1:1 of cyclodextrin molecules to polyphenols is one viable formulation. Broadly speaking, a range of molar ratios of 1:10 to 50:10 of cyclodextrin to polyphenols would be an acceptable formulation. A preferred range of molar ratio of 1:10 to 3:10 cyclodextrin to polyphenols would be an acceptable formulation. A most preferred molar ration of cyclodextrin to polyphenols of from 2:5 to 3:5 would be an acceptable formulation.

Development of an absorption enhancement technology to allow the increase the bioavailability of phenolic compounds will have important positive health effect benefits of the many dietary supplement and pharmaceutical products currently in use. The utility of using trisodium citrate as an agent to enhance drug delivery of polyphenols and thereby improve the efficiency of polyphenols crossing the gut, as shown by the above CaCo2 model and across the blood brain barrier, as shown above with the MDCK model, was further evaluated using these models. Polyphenol extracts of cranberry, blueberry and elderberry containing 30% total phenols, in the presence of trisodium citrate, have shown diffusion enhancement in both models. This data supports more generally the enhanced delivery of the extracts tested, each of which comprised of a wide range of polyphenols varying by sizes, polarities and classes. It is further believed that lower degrees of enrichment of the total phenols (e.g., <10% total phenols) to much higher enrichment of the total phenols (e.g., 35-100% total phenols) will have increased bioavailability from the addition of trisodium citrate. The range of phenolics compounds that will benefit from the addition of trisodium citrate also be from simple monomeric phenolic compounds (e.g., single flavonoids) to highly complex mixtures of polyphenols, including oligomers, phenolic polymers and condensed tannins.

When used as a drug or an over-the-counter formulation of the present disclosure for the treatment and/or prevention of urinary tract infections, or other urogenital conditions, higher purity composition contacting >50% polymeric phenolics from cranberry and blueberry are most preferred, however, a composition containing >35% w/w polymeric phenolics is sufficient. Similarly used as a drug or an over-the-counter formulation of the present disclosure for use as an anti-inflammatory or anti-aging formulation, higher purity composition contacting >50% polymeric phenolics from blueberry are most preferred.

Accordingly, another aspect of the present disclosure is the enhancement of oral uptake of phenolic components in a subject by administering a dose of the enriched, full-spectrum, polyphenolic extract according to the compositions and methods disclosed herein.

In addition, blueberry extracts enriched to contain 30% total phenols, in the presence of β-cyclodextrin, showed marked improved permeation in the gut model described above. This data supports the enhanced delivery of the blueberry extract tested, which consists of a wide range of polyphenols varying by size, polarity and classes. It is further expected that lower degrees of enrichment of a total phenols extract (e.g., <10% total phenols) to much higher enrichment of the total phenols (e.g., 35-100% total phenols) will also have increased bioavailability from the addition of cyclodextrin. The range of phenolics compounds that will benefit from the addition of cyclodextrin ranges from simple monomeric phenolic compounds (e.g., single anthocyanins, flavonoids and the like, as well as phenolic acids) to highly complex mixtures of polyphenols, including oligomers, phenolic polymers and condensed tannins.

Phenolic compounds are well known to be powerful antioxidants, as well as having anti-aging benefits, and are also known to play a preventive role in neurodegenerative, neoplasia, atherosclerosis, cardiovascular, lung and kidney diseases, diabetes and other disorders. The benefit of combining polyphenols with trisodium citrate to increase their bioavailability is expected to have wide utility across a number of diseases, on both their prevention as well as treatment.

In summary, the phenolic polymer extracts and compositions containing the extracts of the present disclosure are characterized by having the capability of exerting enhanced bioactivity, covering a range of in vitro assays, including anti-adhesive assays, ACE2 assays and antiviral assays against controls. The compositions of the present disclosure are further characterized by containing from 5-95% total phenols of high molecular weight by weight of the extracts, which include anthocyanins, glycosides, acylglycosides, polyacylglycosides, metalloanthocyanins and combinations thereof. Flavonoid units of these extracts are expected to comprise catechins, epicatechins, gallocatechins, galloepicatechins and combinations thereof, and optionally phenolic acids, esters, ethers or oxonium derivatives of some of these compounds. Thus, the phenolic compound in the polyphenolic extract may be selected from the group consisting of catechins, epicatechins, proanthocyanidins, gallocatechins, galloepicatechins, anthocyanins, glycosides, acylglycosides, polyacylglycosides, anthocyanidins, phenolic acids of C6-C1 class, benzoic and hydroxybenzoic acids, C6-C3 class of acids, and cinnamic, chlorogenic, and related acids.

The foregoing description is considered as illustrative only of the principles of the disclosure. Further, since numerous modifications and changes will readily occur to those skilled in the art, it is not desired to limit the disclosure to the exact construction and process shown as described above. Accordingly, all suitable modifications and equivalents may be resorted to falling within the scope of the disclosure as defined by the claims that follow.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

1. An enriched, full-spectrum, polyphenolic extract derived from plant material, comprising:
a) phenolic monomers, dimers and trimers;
b) phenolic oligomers; and
c) phenolic polymers;
wherein the polyphenolic extract contains >35% by weight total phenols;
wherein a phenolic compound in the polyphenolic extract is selected from the group consisting of catechins, epicatechins, proanthocyanidins, gallocatechins, galloepicatechins, anthocyanins, glycosides, acylglycosides, polyacylglycosides, anthocyanidins, phenolic acids of C6-C1 class, benzoic and hydroxybenzoic acids, C6-C3 class of acids, and cinnamic, chlorogenic, and related acids; and
wherein at least 10% by weight of the polyphenolic extract are phenolic polymers.

2. The enriched, full-spectrum, polyphenolic extract derived from plant material of claim 1, wherein at least 1% by weight of the polyphenolic extract are anthocyanins.

3. The enriched, full-spectrum, polyphenolic extract derived from plant material of claim 1, wherein at least 5% by weight of the polyphenolic extract are larger than 4.5 kDalton.

4. The enriched, full-spectrum, polyphenolic extract derived from plant material of claim 1, wherein at least 1% by weight of the polyphenolic extract are anthocyanins and at least 5% by weight of the polyphenolic extract are larger than 4.5 kDalton.

5. The enriched, full-spectrum, polyphenolic extract derived from plant material of claim 1, wherein at least 1% by weight of the polyphenolic extract are anthocyanins and at least 5% by weight of the polyphenolic extract are larger than 7.5 kDalton.

6. The enriched, full-spectrum, polyphenolic extract derived from plant material of claim 1, wherein at least 1% by weight of the polyphenolic extract are anthocyanins and at least 5% by weight of the polyphenolic extract are larger than 7.5 kDalton.

7. The enriched, full-spectrum, polyphenolic extract derived from plant material of claim 2, wherein the plant material is selected from the group consisting of blueberries, cranberries and elderberries.

8. The enriched, full-spectrum, polyphenolic extract derived from plant material of claim 1, wherein the plant material is selected from the group consisting of blueberries, cranberries and elderberries.

9. The enriched, full-spectrum, polyphenolic extract derived from plant material of claim 1, wherein the plant material is selected from the group consisting of blueberries, bilberries, elderberries, plums, blackberries, strawberries, red currants, black currants, cranberries, cherries, red raspberries, black raspberries, grapes, pomegranates, gooseberries and currants.

10. The enriched, full-spectrum, polyphenolic extract derived from plant material of claim 1, wherein the plant material is selected from the group consisting of hibiscus flowers, bell peppers, beans, peas, red cabbage, purple corn, violet sweet potatoes, olives, mangosteen, apples, hawthorn, and oranges.

11. The enriched, full-spectrum, polyphenolic extract derived from plant material of claim 1, wherein the extract is combined with trisodium citrate, creating thereby an oral composition having enhanced bioavailability.

12. The enriched, full-spectrum, polyphenolic extract derived from plant material of claim 1, wherein the extract is combined with β-cyclodextrin, creating thereby an oral composition having enhanced bioavailability.

13. The enriched, full-spectrum, polyphenolic extract derived from plant material of claim 8, wherein the extract is combined with trisodium citrate, creating thereby an oral composition having enhanced bioavailability.

14. The enriched, full-spectrum, polyphenolic extract derived from plant material of claim 8, wherein the extract is combined with β-cyclodextrin, creating thereby an oral composition having enhanced bioavailability.

15. A method of manufacturing an enriched, full-spectrum, polyphenolic extract derived from plant material, comprising:
a) extracting and filtering juice from the plant material to obtain a raw extract;
b) diluting the raw extract with acidified water to a juice strength extract;
c) passing the juice strength extract through a PS-DVD crosslinked porous gel packed column to cause phenolic constituents in the juice strength extract to adhere to the column and non-phenolic contaminants to pass through the gel-packed column;
d) washing the phenolic constituents adhering to the column with at least 3 to 20 column volumes of acidified water to remove sugars and organic acids to remove non-phenolic contaminants adsorbed/embedding in the phenolic constituents;
e) eluting and collecting the phenolic constituents adhering to the gel column with lower alcohol/acidified water in the range of 50-90% to create a semi-purified polyphenolic extract;
f) concentrating the semi-purified polyphenolic extract by removing ethanol and water therefrom and diluting the concentrated product with acidified water to an approximate volume of the juice strength extract to create a semi-purified eluent;
g) loading the semi-purified eluent onto a second PS-DVD crosslinked porous gel packed column so as to cause phenolic constituents in the juice strength extract to adhere to the column and additional non-phenolic contaminants to pass through the gel-packed column;
h) washing the phenolic constituents adhering to the column with at least 3 to 20 column volumes of acidified water to remove additional sugars, organic acids and non-phenolic contaminants adsorbed/embedding in the phenolic constituents;
i) eluting the adhered polyphenols from the gel column with acidified alcohols containing 1-3 carbons to and collecting the eluent into an enriched polyphenolic eluent; and
j) concentrating the enriched polyphenolic eluent removing liquid therefrom to obtain an enriched, full-spectrum, polyphenolic extract containing >35% total phenols and at least 10% by weight of the polyphenolic extract are >2.5 kDalton.

16. A method of manufacturing an enriched, full-spectrum, polyphenolic extract derived from plant material, comprising:
a) extracting and filtering juice from the plant material to obtain a raw extract;
b) diluting the raw extract with acidified water to a juice strength extract;
c) passing the juice strength extract through a mixed resin PS-DVD crosslinked porous gel packed column to cause phenolic constituents in the juice strength extract to adhere to the column and non-phenolic contaminants to pass through the gel-packed column;
d) washing the phenolic constituents adhering to the column with at least 3 to 20 column volumes of acidified water to remove sugars and organic acids to remove non-phenolic contaminants adsorbed/embedding in the phenolic constituents;
e) eluting and collecting the phenolic constituents adhering to the gel column with lower alcohol/acidified water in 50-90% to create a semi-purified polyphenolic extract;
f) concentrating the semi-purified polyphenolic extract by removing ethanol and water therefrom and diluting the concentrated product with acidified water to an approximate volume of the juice strength extract to create a semi-purified eluent;
g) eluting the adhered polyphenols from the gel column with acidified alcohols containing 1-3 carbons to and collecting the eluent into an enriched polyphenolic eluent; and
h) concentrating the enriched polyphenolic eluent removing liquid therefrom to obtain an enriched, full-spectrum, polyphenolic extract containing >35% total phenols and at least 10% by weight of the polyphenolic extract are >2.5 kDalton.

17. A method of manufacturing an enriched, full-spectrum, polyphenolic extract derived from plant material, comprising:
a) extracting and filtering juice from the plant material to obtain a raw extract;
b) diluting the raw extract with acidified water to a juice strength extract;
c) passing the juice strength extract through individual PS-DVD crosslinked porous gel packed column to cause phenolic constituents in the juice strength extract to adhere to the column and non-phenolic contaminants to pass through the gel-packed column;
d) washing the phenolic constituents adhering to the column with at least 3 to 25 column volumes of acidified water to remove sugars and organic acids to remove non-phenolic contaminants adsorbed/embedding in the phenolic constituents;
e) eluting and collecting the phenolic constituents adhering to the gel column with lower alcohol/acidified water in 50-90% to create a semi-purified polyphenolic extract;
f) concentrating the semi-purified polyphenolic extract by removing ethanol and water therefrom and diluting the concentrated product with acidified water to an approximate volume of the juice strength extract to create a semi-purified eluent;
g) eluting the adhered polyphenols from the gel column with acidified alcohols containing 1-3 carbons to and collecting the eluent into an enriched polyphenolic eluent; and
h) concentrating the enriched polyphenolic eluent removing liquid therefrom to obtain an enriched, full-spectrum, polyphenolic extract containing >35% total phenols and at least 10% by weight of the polyphenolic extract are >2.5 kDalton.

18. The method of claim 15, 16 or 17 wherein the plant material is selected from the group consisting of blueberries, cranberries, and elderberries.

19. The method of claims 15 wherein the plant material is selected from the group consisting of blueberries, bilberries, elderberries, plums, blackberries, strawberries, red currants, black currants, cranberries, cherries, red raspberries, black raspberries, grapes, gooseberries, pomegranates, and currants.

20. The method of claims 15 wherein the plant material is selected from the group consisting of hibiscus flowers, bell peppers, beans, peas, red cabbage, purple corn, violet sweet potatoes, olives, mangosteen, apples, hawthorn, and oranges.

21. The method of claims 15 further comprising the step of:
combining the enriched, full-spectrum, polyphenolic extract containing >35% total phenols with trisodium citrate to produce an oral composition exhibiting enhanced bioavailability.

22. The method of claims 15 further comprising the step of:
combining the enriched, full-spectrum, polyphenolic extract containing >35% total phenols with cyclodextrin to produce an oral composition exhibiting enhanced bioavailability.

23. The method of manufacturing the enriched, full-spectrum, polyphenolic extract derived from plant material of claims 15 wherein the individual process yields an enriched, full-spectrum, polyphenolic extract containing from >35% total phenols to 95% total phenols.

24. A method of enhancing oral uptake of phenolic components in a subject, comprising administering to an individual a dose of the enriched, full-spectrum, polyphenolic extract according to claim 11.

25. A method of enhancing oral uptake of phenolic components in a subject, comprising administering to an individual a dose of the enriched, full-spectrum, polyphenolic extract according to claim 12.

26. A method of enhancing oral uptake of phenolic components in a subject, comprising administering to an individual a dose of the enriched, full-spectrum, polyphenolic extract made according to the method of claims 15 combined with trisodium citrate.

27. A method of enhancing oral uptake of phenolic components in a subject, comprising administering to an individual a dose of the enriched, full-spectrum, polyphenolic extract made according to the method of claims 15, comprising the step of combining the polyphenolic extract with β-cyclodextrin.

28. The enriched, full-spectrum, polyphenolic extract derived from plant material of claim 1, wherein the plant material is obtained from a Vaccinium species.

29. The enriched, full-spectrum, polyphenolic extract derived from plant material of claim 28, wherein the plant material is obtained from Vaccinium macrocarpon.

30. The enriched, full-spectrum, polyphenolic extract derived from plant material of claim 28, wherein the plant material is obtained from Vaccinium angustifolium.

31. The enriched, full-spectrum, polyphenolic extract derived from plant material of claim 1, wherein the plant material is obtained from a Sambucus species.

32. The enriched, full-spectrum, polyphenolic extract derived from plant material of claim 31, wherein the plant material is obtained from Sambucus nigra.

33. The enriched, full-spectrum, polyphenolic extract derived from plant material of claim 31, further comprising:
trisodium citrate,
wherein the molar ratio of trisodium citrate to phenols in the polyphenolic extract is from 1:10 to 1:200.

34. The enriched, full-spectrum, polyphenolic extract derived from plant material of claim 31, wherein the molar ratio of trisodium citrate to phenols in the polyphenolic extract is from 1:10 to 1:20.

35. The enriched, full-spectrum, polyphenolic extract derived from plant material of claim 31, further comprising:
β-cyclodextrin
wherein the molar ratio of trisodium citrate to phenols in the polyphenolic extract is from 1:10 to 3:10.

36. Then enriched polyphenolic extract derived from plant material according to claim 1, wherein the phenolic oligomers are selected from the group consisting of oligomers of from 1.2 kDalton through 3 kDalton; and
the phenolic polymers selected from the group consisting of polymers of from 3 kDalton through 12 kDalton.

37. The enriched polyphenolic extract derived from plant material of claim 36, wherein at least 1% by weight of the polyphenolic extract are anthocyanins.

38. The enriched polyphenolic extract derived from plant material of claim 36, wherein at least 5% by weight of the polyphenolic extract are larger than 4.5 kDalton.

39. The enriched polyphenolic extract derived from plant material of claim 36, wherein at least 1% by weight of the polyphenolic extract are anthocyanins and at least 5% by weight of the polyphenolic extract are larger than 4.5 kDalton.

40. The enriched polyphenolic extract derived from plant material of claim 36, wherein at least 5% by weight of the polyphenolic extract are larger than 7.5 kDalton.

41. The enriched polyphenolic extract derived from plant material of claim 36, wherein at least 1% by weight of the polyphenolic extract are anthocyanins and at least 5% by weight of the polyphenolic extract are larger than 7.5 kDalton.

42. The enriched polyphenolic extract derived from plant material of claim 37, wherein the plant material is selected from the group consisting of blueberries, cranberries and elderberries.

43. The enriched polyphenolic extract derived from plant material of claim 36, wherein the plant material is selected from the group consisting of blueberries, cranberries and elderberries.

44. The enriched polyphenolic extract derived from plant material of claim 36, wherein the plant material is selected from the group consisting of blueberries, bilberries, elderberries, plums, blackberries, strawberries, red currants, black currants, cranberries, cherries, red raspberries, black raspberries, grapes, pomegranates, gooseberries and currants.

45. The enriched polyphenolic extract derived from plant material of claim 36, wherein the plant material is selected from the group consisting of hibiscus flowers, bell peppers, beans, peas, red cabbage, purple corn, violet sweet potatoes, olives, mangosteen, apples, hawthorn, and oranges.

46. The enriched polyphenolic extract derived from plant material of claim 36, wherein the extract is combined with trisodium citrate, creating thereby an oral composition having enhanced bioavailability.

47. The enriched polyphenolic extract derived from plant material of claim 36, wherein the extract is combined with β-cyclodextrin, creating thereby an oral composition having enhanced bioavailability.

48. The enriched polyphenolic extract derived from plant material of claim 44, wherein the extract is combined with trisodium citrate, creating thereby an oral composition having enhanced bioavailability.

49. The enriched polyphenolic extract derived from plant material of claim 44, wherein the extract is combined with β-cyclodextrin, creating thereby an oral composition having enhanced bioavailability.

50. A method of preventing or treating urogenital infections in a mammal comprising orally administering to the mammal a dose of the of the enriched polyphenolic extract of claim 36, wherein the dose includes at least 35% total phenols which includes polar and non-polar enriched phenols.

51. The method of claim 50, wherein the enriched phenolic extract is derived from fruits selected from the group consisting of cranberries, blueberries and elderberries.

52. A method of supplementing the diet of a mammal comprising orally administering to the mammal the enriched polyphenolic extract of claim 36, wherein the dose includes at least 35% total phenols which includes polar and non-polar enriched phenols.

53. The method of claim 52, wherein the enriched polyphenolic extract is derived from fruits selected from the group consisting of cranberries, blueberries and elderberries.

54. A method of use for enhancing uptake of phenolic components across the blood brain barrier in a subject, comprising administering to an individual a dose of:
at least one phenolic compound; and
a compound selected from the group consisting of a citrate and pharmaceutically acceptable salts thereof.
